(19) United States
(12) Patent Application Publication
(10) Pub. No.: US 2024/0270055 A1
(43) Pub. Date: Aug. 15, 2024

(54) HEATER ELEMENT AND VEHICLE COMPARTMENT PURIFICATION SYSTEM

(71) Applicant: NGK INSULATORS, LTD., Nagoya-City (JP)

(72) Inventors: Yukio MIYAIRI, Nagoya-Shi (JP); Masaaki MASUDA, Nagoya-Shi (JP); Takuya NAKASHIMA, Nagoya-Shi (JP)

(73) Assignee: NGK INSULATORS, LTD., Nagoya-City (JP)

(21) Appl. No.: 18/643,063

(22) Filed: Apr. 23, 2024

Related U.S. Application Data
(63) Continuation of application No. PCT/JP2022/035207, filed on Sep. 21, 2022.

(30) Foreign Application Priority Data
Oct. 29, 2021 (JP) 2021-178100

(51) Int. Cl. B60H 3/06; B01D 53/04; B01D 53/86; B01D 53/96; B01J 35/57; B60H 1/00; B60H 1/22; F24H 3/04; H05B 3/12

(52) U.S. Cl. CPC B60H 3/0608; B01D 53/0438; B01D 53/86; B01D 53/96; B01J 35/57; B60H 1/00849; B60H 1/2225; F24H 3/0429; B01D 2253/3425; B01D 2257/504; B01D 2257/708; B01D 2257/80; B01D 2257/90; B01D 2259/4009; B01D 2259/4566; B60H 2003/0691; F24H 2250/04; H05B 3/12

(57) ABSTRACT

A heater element includes a honeycomb structure, and satisfies either of the following condition (i) or (ii): (i) a first electrode is provided on one end surface, and a second electrode has an electrode portion A provided on the other end surface, and an electrode portion B connected to the electrode portion A and provided on a surface of partition walls; (ii) the first electrode has an electrode portion A provided on the one end surface, and an electrode portion B connected to the electrode portion A and provided on the surface of the partition walls, and the second electrode has an electrode portion A provided on the other end surface, and an electrode portion B connected to the electrode portion A and provided on a surface of the partition walls.

### FIELD OF THE INVENTION

The present invention relates to a heater element and a vehicle compartment purification system.

### BACKGROUND OF THE INVENTION

In various vehicles such as automobiles, there is an increasing demand for improvement of vehicle compartment. Specific requirements include reducing CO₂ in the vehicle compartment to suppress driver drowsiness, controlling the humidity in the vehicle compartment, and eliminating harmful volatile components such as odor components and allergy-inducing components in the vehicle compartment, and the like. Ventilation can be mentioned as an effective measure to meet such demands, but ventilation causes a large loss of heater energy in winter and causes deterioration of energy efficiency in winter. In particular, in an electric vehicle (BEV: Battery Electric Vehicle), there is a problem that the cruising range is significantly reduced due to the energy loss.

As methods for solving the above problem, Patent Literature 1 and Patent Literature 2 disclose a vehicle compartment purification system which captures components to be removed such as water vapor and CO₂ in the air of a vehicle compartment by a functional material such as an adsorbent, and react or separate the components to be removed by heating and release them to the outside of the vehicle to regenerate the functional material. In such a vehicle compartment purification system, it is required that the air and the functional materials come into contact with each other as much as possible in order to secure the capture performance of the component to be removed, and that the functional material can be heated to a predetermined temperature in order to promote the regeneration of the functional material. Regeneration is performed, for example, by a method of removing the substance adsorbed on the functional material by an oxidation reaction, and a method of desorbing the substance adsorbed on the functional material and discharging the substance. However, in any case, it is necessary to heat the functional material to an appropriate temperature according to the adsorbed substance.

On the other hand, Patent Literature 3 discloses a heater element, comprising a pillar-shaped honeycomb structure portion having an outer peripheral side wall, and partition walls provided inside the outer peripheral side wall, the partition walls partitions a plurality of cells forming flow paths from a first end surface to a second end surface, wherein the partition walls have PTC characteristics, an average thickness of the partition walls is 0.13 mm or less, and an open frontal area on the first and second end surfaces is 0.81 or more. This heater element is used for a heater for heating a vehicle compartment.

### PRIOR ART

### Patent Literature

[Patent Literature 1] Japanese Patent Application Publication No. 2020-104774
[Patent Literature 2] Japanese Patent Application Publication No. 2020-111282
[Patent Literature 3] WO 2020/036067

### SUMMARY OF THE INVENTION

The heater element described in Patent Literature 3 is used for heating a vehicle compartment, and it is an efficient heating means because it has a honeycomb structure and can increase the heating area. Therefore, it is considered that the use of such a heater element as a carrier of the functional material can contribute to shortening the regeneration time of the functional material.

In particular, since the heater element described in Patent Literature 3 can be heated by energization and has PTC characteristics, it is considered the functional material can be easily heated, while excessive heat generation can be suppressed and thermal deterioration of the functional material can be suppressed. In addition, since the risk of excessive temperature is avoided, safety can be ensured even if the initial resistance is set small and the heating rate is increased, and the temperature can be raised in a short time.

However, as a result of the study of the present inventors, when a functional material-containing layer is provided on the surface of the partition walls partitioning the cells of the heater element described in Patent Literature 3, it is difficult for the temperature to rise near the inlet side of the heater element, and it has been found that the area in the direction in which the flow paths extend where the functional material can be effectively heated within the cell becomes narrower. In other words, part of the functional material carried on the heater element has low regeneration efficiency and cannot be utilized effectively. Further, when the functional material is a catalyst, heating may be necessary to activate the catalyst, but if the temperature of the carried catalyst is insufficiently raised, the catalyst cannot be utilized effectively. Providing a functional material-containing layer that cannot be utilized effectively becomes a factor that reduces the cost performance of the heater element.

The present invention has been created in view of the above circumstances, and in one embodiment, an object is to provide a heater element that can widen the area in the direction in which the flow paths extend where a functional material can be effectively heated. Further, in another embodiment of the present invention, an object is to provide a vehicle compartment purification system equipped with such a heater element. In yet another embodiment of the present invention, an object is to provide a vehicle compartment purification system that is beneficial for increasing the rate at which functional materials can be utilized effectively.

### [Aspect 1]

A heater element, comprising:
a honeycomb structure comprising an outer peripheral wall and partition walls provided on an inner side of the outer peripheral wall, the partition walls partitioning a plurality of cells that form flow paths extending from one end surface to an other end surface, wherein at least the partition walls are made of a material having PTC characteristics; and
a pair of electrodes composed of a first electrode and a second electrode;
wherein the first electrode and the second electrode satisfy either of the following condition (i) or (ii):
(i) the first electrode is provided on the one end surface, and
   the second electrode comprises an electrode portion A provided on the other end surface, and an electrode portion B connected to the electrode portion A and provided on a surface of the partition walls over a predetermined length from the other end surface in a direction in which the flow paths extend;
(ii) the first electrode comprises an electrode portion A provided on the one end surface, and an electrode portion B connected to the electrode portion A and provided on the surface of the partition walls over a predetermined length from the one end surface in the direction in which the flow paths extend, and
   the second electrode comprises an electrode portion A provided on the other end surface, and an electrode portion B connected to the electrode portion A and provided on a surface of the partition walls over a predetermined length from the other end surface in a direction in which the flow paths extend.

### [Aspect 2]

The heater element according to aspect 1, wherein the predetermined length of the electrode portion B has an average length of 1/200 or more and less than ½ of a length of the honeycomb structure in the direction in which the flow paths extend.

### [Aspect 3]

The heater element according to aspect 1 or 2, wherein the electrode portion B is continuously provided over the predetermined length over the entire surface of all the partition walls partitioning the plurality of cells.

### [Aspect 4]

The heater element according to aspect 1 or 2, wherein the electrode portion B is continuously provided over the predetermined length on a part of the surface of the partition walls partitioning the plurality of cells.

### [Aspect 5]

The heater element according to any one of aspects 1 to 4, wherein the material having PTC characteristics is composed of a material containing barium titanate as a main component and substantially free of lead.

### [Aspect 6]

The heater element according to any one of aspects 1 to 5, wherein a volume resistivity at 25° C. of the material having PTC characteristics is 0.5 Ω·cm or more and 20 Ω·cm or less.

### [Aspect 7]

The heater element according to any one of aspects 1 to 6, wherein an average thickness of the electrode portion B is 1/10,000 or more and 1/10 or less of a hydraulic diameter of the cells.

### [Aspect 8]

The heater element according to any one of aspects 1 to 7, wherein in the honeycomb structure, a thickness of the partition walls is 0.125 mm or less, a cell density is 100 cells/cm² or less, and a cell pitch is 1.0 mm or more.

### [Aspect 9]

The heater element according to any one of aspects 1 to 7, wherein in the honeycomb structure, a thickness of the partition walls is 0.08 mm or more and 0.36 mm or less, a cell density is 2.54 cells/cm² or more and 140 cells/cm² or less, and an open frontal area of the cells is 0.70 or more.

### [Aspect 10]

The heater element according to aspect any one of aspects 1 to 9, wherein the first electrode and the second electrode are made of the same material.

### [Aspect 11]

The heater element according to any one of aspects 1 to 10, further comprising a functional material-containing layer on the surface of the partition walls.

### [Aspect 12]

The heater element according to aspect 11, wherein the functional material-containing layer contains a functional material having a function of adsorbing one or more selected from water vapor, carbon dioxide, and odor components.

### [Aspect 13]

The heater element according to aspect 11 or 12, wherein the functional material-containing layer comprises a catalyst.

### [Aspect 14]

A vehicle compartment purification system, comprising:
at least one heater element according to any one of aspects 1 to 13;
a power supply for applying voltage to the heater element;
an inflow piping that communicates the vehicle compartment with an inlet end surface of the heater element;
an outflow piping having a first path that communicates an outlet end surface of the heater element with the vehicle compartment;
a ventilator for causing air from the vehicle compartment to flow into the inlet end surface of the heater element via the inflow piping;
wherein the heater element is arranged such that the inlet end surface is the one end surface and the outlet end surface is the other end surface, or such that the inlet end surface is the other end surface, and the outlet end surface is the one end surface.

### [Aspect 15]

The vehicle compartment purification system according to aspect 14, wherein the heater element is arranged such that the inlet end surface is the one end surface and the outlet end surface is the other end surface.

### [Aspect 16]

The vehicle compartment purification system according to aspect 14 or 15,
wherein in addition to the first path, the outflow piping comprises a second path that communicates the outlet end surface of the heater element with an outside of the vehicle, and
the outflow piping comprises a switching valve that is configured to switch a flow of air flowing through the outflow piping between the first path and the second path;
wherein the vehicle compartment purification system further comprises a controller capable of switching between:
a first mode in which the applied voltage from the power supply is turned off, the switching valve is switched such that the air flowing through the outflow piping passes through the first path, and the ventilator is turned on, and
a second mode in which the applied voltage from the power supply is turned on, the switching valve is switched such that the air flowing through the outflow piping passes through the second path, and the ventilator is turned on.

### [Aspect 17]

The vehicle compartment purification system according to any one of aspects 14 to 16, wherein an additive functional body is provided downstream and adjacent to the heater element, the additive functional body comprising:
a honeycomb structure comprising an outer peripheral wall and partition walls provided on an inner side of the outer peripheral wall, the partition walls partitioning a plurality of cells that form flow paths extending from an inlet end surface to an outlet end surface; and
a functional material-containing layer provided on a surface of the partition walls.

### [Aspect 18]

The vehicle compartment purification system according to aspect 17, wherein at least the partition walls of the additive functional body is made of cordierite.

### [Aspect 19]

A vehicle compartment purification system, comprising:
a heater element comprising:
   a honeycomb structure comprising an outer peripheral wall and partition walls provided on an inner side of the outer peripheral wall, the partition walls partitioning a plurality of cells that form flow paths extending from an inlet end surface to an outlet end surface;
   a first electrode provided on the inlet end surface; and
   a second electrode provided on the outlet end surface;
an additive functional body provided downstream and adjacent to the heater element, the additive functional body comprising:
   a honeycomb structure comprising an outer peripheral wall and partition walls provided on an inner side of the outer peripheral wall, the partition walls partitioning a plurality of cells that form flow paths extending from an inlet end surface to an outlet end surface; and
   a functional material-containing layer provided on a surface of the partition walls;
a power supply for applying voltage to the heater element;
an inflow piping that communicates the vehicle compartment with the inlet end surface of the heater element;
an outflow piping comprising a first path that communicates the outlet end surface of the additive functional body with the vehicle compartment; and
a ventilator for causing air from the vehicle compartment to flow into the inlet end surface of the heater element via the inflow piping.

According to one embodiment of the present invention, a heater element that can widen the area in the direction in which the flow paths extend where a functional material can be effectively heated is provided. Further, according to another embodiment of the present invention, a vehicle compartment purification system comprising the heater element is provided. By providing a functional material-containing layer on the surface of the partition walls of the heater element, it is possible to reduce the proportion of functional materials that are difficult to regenerate and cannot be utilized effectively and/or functional materials that hardly demonstrate their functions due to insufficient heating and cannot be utilized effectively. In other words, the area of functional material-containing layers that can be utilized effectively is expanded. This makes it possible to improve the cost performance of a heater element.

Furthermore, in the vehicle compartment purification system according to yet another embodiment of the present invention in which the heater element is arranged on the upstream side and an additive functional body is arranged on the downstream side, it is possible to increase the ratio of functional materials that can be utilized effectively.

### BRIEF DESCRIPTION OF THE DRAWINGS

FIG. 1A is a schematic perspective view of a heater element according to a first embodiment of the present invention, viewed from one end surface.

FIG. 1B is a schematic perspective view of the heater element according to the first embodiment of the present invention, viewed from the other end surface.

FIG. 1C is a schematic diagram of a cross section parallel to the flow path direction passing through a central axis O extending in the flow path direction of the heater element according to the first embodiment of the present invention.

FIG. 1D is a schematic diagram of a cross section orthogonal to the flow path direction when the heater element according to the first embodiment of the present invention is cut along the line X-X in FIG. 1C.

FIG. 2A is a schematic perspective view of a heater element according to a second embodiment of the present invention, viewed from one end surface.

FIG. 2B is a schematic perspective view of a heater element according to the second embodiment of the present invention, viewed from the other end surface.

FIG. 2C is a schematic diagram of a cross section parallel to the flow path direction passing through the central axis O extending in the flow path direction of a heater element according to the second embodiment of the present invention.

FIG. 2D is a schematic diagram of a cross section orthogonal to the flow path direction when the heater element according to the second embodiment of the present invention is cut along the line X-X in FIG. 2C.

FIG. 3A is a schematic diagram of a cross section orthogonal to the flow path direction of a heater element according to another embodiment of the present invention.

FIG. 3B is a schematic diagram of a cross section orthogonal to the flow path direction of a heater element according to another embodiment of the present invention.

FIG. 3C is a schematic diagram of a cross section orthogonal to the flow path direction of a heater element according to another embodiment of the present invention.

FIG. 3D is a schematic diagram of a cross section orthogonal to the flow path direction of a heater element according to another embodiment of the present invention.

FIG. 4 is a schematic diagram showing the configuration of a vehicle compartment purification system according to an embodiment of the present invention.

FIG. 5 is a schematic diagram showing the configuration of a vehicle compartment purification system according to another embodiment of the present invention.

FIG. 6A is a schematic perspective view of an example of the additive functional body when viewed from one end surface.

FIG. 6B is a schematic diagram of a cross section parallel to the flow path direction passing through the central axis O extending in the flow path direction of the additive functional body shown in FIG. 6A.

FIG. 6C is a schematic diagram of a cross section of the additive functional body taken along line X-X in FIG. 6B, which is orthogonal to the flow path direction.

FIG. 7A is a schematic perspective view of an example of a heater element that can be used in a vehicle compartment purification system according to yet another embodiment of the present invention, when viewed from one end surface.

FIG. 7B is a schematic diagram of a cross section parallel to the flow path direction passing through the central axis O extending in the flow path direction of the heater element shown in FIG. 7A.

FIG. 7C is a schematic diagram of a cross section of the heater element taken along line X-X in FIG. 7B, which is orthogonal to the flow path direction.

FIG. 8 is a schematic diagram showing the configuration of a vehicle compartment purification system according to yet another embodiment of the present invention.

FIG. 9 is a contour diagram showing the temperature distribution inside the heater element based on simulation.

### DETAILED DESCRIPTION OF THE INVENTION

Hereinafter, embodiments of the present invention will now be described in detail with reference to the drawings. It should be understood that the present invention is not intended to be limited to the following embodiments, and any change, improvement or the like of the design may be appropriately added based on ordinary knowledge of those skilled in the art without departing from the spirit of the present invention.

### (1. Heater Element)

The heater element according to one embodiment of the present invention can be suitably used as a heater element used in a vehicle compartment purification system in various vehicles such as automobiles. Vehicles are not particularly limited, and examples thereof include automobiles and electric trains. Examples of automobiles include, but are not limited to, gasoline-powered vehicles, diesel-powered vehicles, gas-fueled vehicles using CNG (Compressed Natural Gas), LNG (Liquefied Natural Gas), fuel cell vehicles, electric vehicles, and plug-in hybrid vehicles. The heater element according to the embodiments of the present invention can be particularly suitably used for a vehicle having no internal combustion engine such as an electric vehicle and an electric train.

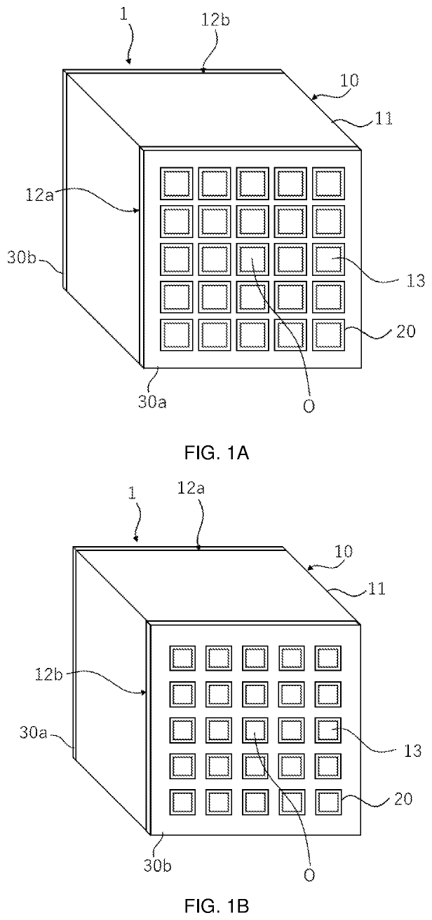

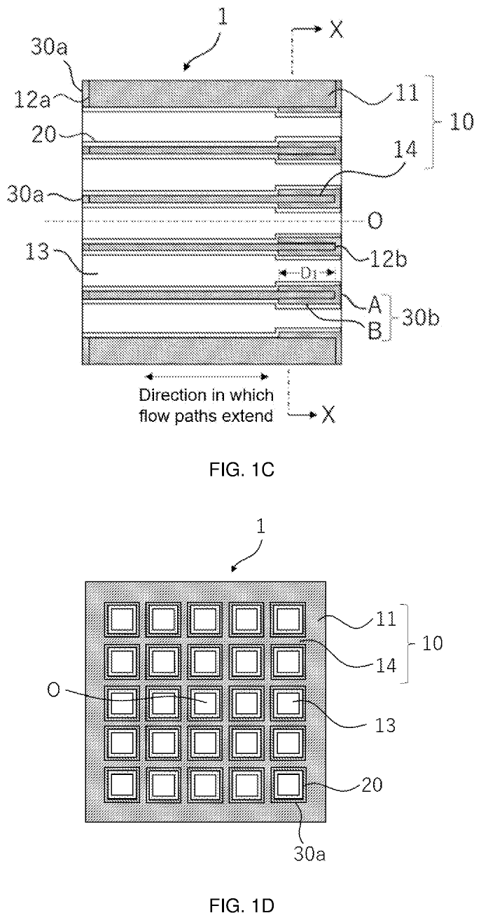

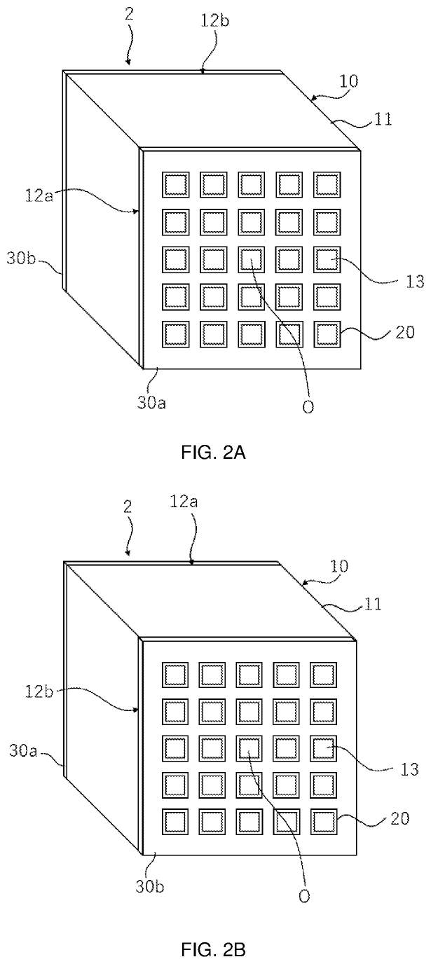

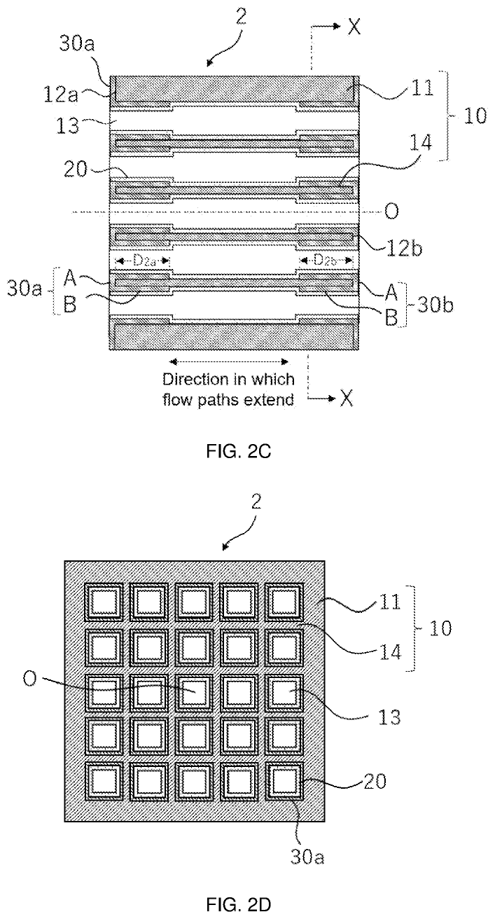

FIGS. 1A to 1D show schematic perspective views and cross-sectional views of a heater element 1 according to a first embodiment of the present invention. FIGS. 2A to 2D show schematic perspective views and cross-sectional views of a heater element 2 according to a second embodiment of the present invention. As shown in FIGS. 1A to 1D and 2A to 2D, the heater element 1, 2 comprises a honeycomb structure 10 having an outer peripheral wall 11 and partition walls 14 provided on an inner side of the outer peripheral wall 11, the partition walls 14 partitioning a plurality of cells 13 that form flow paths extending from one end surface 12a to another end surface 12b. The heater element 1, 2 comprises a pair of electrodes composed of a first electrode 30a and a second electrode 30b. Furthermore, the heater element 1, 2 may comprise a functional material-containing layer 20 provided on the surface of the partition walls 14. Each component of the heater element 1, 2 will be described in detail below.

### (1-1 Honeycomb Structure)

The shape of the honeycomb structure 10 is not particularly limited. For example, the outer shape of the cross-section orthogonal to the flow path direction (direction in which the cells 13 extend) of the honeycomb structure 10 can be polygonal (quadrangle (rectangle, square), pentagon, hexagon, heptagon, octagon, and the like), circular, oval (egg-shape, ellipse, oblong, rounded rectangle, and the like). In addition, the end surfaces (the one end surface 12a and the end surface 12b) have the same shape as the cross-section. When the cross section and the end surfaces are polygonal, the corners may be chamfered.

The shape of the cells 13 is not particularly limited, and in the cross-section of the honeycomb structure 10 orthogonal to the flow path direction, it can be polygonal (quadrangle, pentagon, hexagon, heptagon, octagon, and the like), circular, or oval. The shapes may be uniform or may be a combination of two or more. Further, among these shapes, a quadrangle or a hexagon is preferable. By providing the cells 13 having such a shape, it is possible to reduce the pressure loss when the air flows. In addition, FIGS. 1 and 2 show, as an example, a honeycomb structure 10 in which the outer shape of the cross-section and the shape of the cells 13 are quadrangular in the cross-section orthogonal to the flow path direction.

The honeycomb structure 10 may be a honeycomb joint body having a plurality of honeycomb segments and joining layers for joining between the outer peripheral side surfaces of the plurality of honeycomb segments. By using the honeycomb joint body, it is possible to increase the total cross-sectional area of the cells 13, which is important for securing the air flow velocity, while suppressing the occurrence of cracks.

In addition, the joining layers can be formed by using a joining material. The joining material is not particularly limited, but a ceramic material to which a solvent such as water is added to form a paste can be used. The joining material may contain a material having PTC (Positive Temperature Coefficient) characteristics, or may contain the same material as the outer peripheral wall 11 and the partition walls 14. In addition to the role of joining the honeycomb segments to each other, the joining material can also be used as an outer peripheral coating material after joining the honeycomb segments.

From the viewpoints of ensuring the strength of the honeycomb structure 10, reducing the pressure loss when air passes through the cells 13, securing the amount of the functional material carried, and securing the contact area with the air flowing in the cells 13, and the like, it is desirable to preferably combine the thickness of the partition walls 14, the cell density, and the cell pitch (or the open frontal area of the cells).

In the present specification, the thickness of the partition wall 14 refers to a crossing length of a line segment that crosses the partition wall 14 when the centers of gravity of adjacent cells 13 are connected by this line segment in a cross-section orthogonal to the flow path direction. The thickness of the partition walls 14 refers to the average value of the thicknesses of all the partition walls 14.

In the present specification, the cell density is a value obtained by dividing the number of cells by the area of one end surface of the honeycomb structure 10 (the total area of the partition walls 14 and the cells 13 excluding the outer peripheral wall 11).

In the present specification, the cell pitch refers to a value obtained by the following calculation. First, the area per cell is calculated by dividing the area of one end surface of the honeycomb structure 10 (the total area of the partition wall 14 and the cells 13, excluding the outer peripheral wall 11) by the number of cells. Next, the square root of the area per cell is calculated, and this is deemed as the cell pitch.

In the present specification, the open frontal area of the cells is a value obtained by dividing the total area of the cells 13 partitioned by the partition walls 14 in a cross-section orthogonal to the flow path direction of the honeycomb structure 10 by the area of one end surface (the total area of the partition walls 14 and the cells 13, excluding the outer peripheral wall 11). In addition, in calculating the open frontal area of the cells 13, the second electrode 30b, and the functional material-containing layer 20 are not taken into consideration.

In an embodiment advantageous from the viewpoint of carrying a sufficient amount of functional material, the thickness of the partition walls is 0.125 mm or less, the cell density is 100 cells/cm² or less, and the cell pitch is 1.0 mm or more. In a preferred embodiment, the thickness of the partition walls is 0.100 mm or less, the cell density is 70 cells/cm² or less, and the cell pitch is 1.2 mm or more. In a more preferable embodiment, the thickness of the partition walls is 0.080 mm or less, the cell density is 65 cells/cm² or less, and the cell pitch is 1.3 mm or more.

In each of the above embodiments, from the viewpoint of ensuring the strength of the honeycomb structure and keeping the electrical resistance low, the lower limit of the thickness of the partition walls is preferably 0.010 mm or more, more preferably 0.020 mm or more, and even more preferably 0.030 mm or more.

In each of the above embodiments, from the viewpoint of ensuring the strength of the honeycomb structure, keeping the electric resistance low, and increasing the surface area to promote the reaction, adsorption, and detachment, the lower limit of the cell density is preferably 30 cells/cm² or more, more preferably 35 cells/cm² or more, and even more preferably 40 cells/cm² or more.

In each of the above embodiments, from the viewpoint of ensuring the strength of the honeycomb structure, keeping the electric resistance low, and increasing the surface area to promote the reaction, adsorption, and detachment, the upper limit of the cell pitch is preferably 2.0 mm or less, more preferably 1.8 mm or less, and even more preferably 1.6 mm or less.

In an embodiment advantageous from the viewpoint of reducing pressure loss and maintaining strength, the thickness of the partition walls is 0.08 mm or more and 0.36 mm or less, and the cell density is 2.54 cells/cm² or more and 140 cells/cm² or less, and the open frontal area of the cells is 0.70 or more. In a preferred embodiment, the thickness of the partition walls is 0.09 mm or more and 0.35 mm or less, the cell density is 15 cells/cm² or more and 100 cells/cm² or less, and the open frontal area of the cells is 0.80 or more. In a more preferable embodiment, the thickness of the partition walls is 0.14 mm or more and 0.30 mm or less, the cell density is 20 cells/cm² or more and 90 cells/cm² or less, and the open frontal area of the cells is 0.85 or more.

In each of the above embodiments, from the viewpoint of ensuring the strength of the honeycomb structure, the upper limit of the open frontal area of the cells is preferably 0.94 or less, more preferably 0.92 or less, and even more preferably 0.90 or less.

The thickness of the outer peripheral wall 11 is not particularly limited, but is preferably determined based on the following viewpoints. First, from the viewpoint of reinforcing the honeycomb structure 10, the thickness of the outer peripheral wall 11 is preferably 0.05 mm or more, more preferably 0.06 mm or more, even more preferably 0.08 mm or more. On the other hand, from the viewpoint of increasing the electrical resistance to suppress the initial current and reducing the pressure loss when air flows, the thickness of the outer peripheral wall 11 is preferably 1.0 mm or less, more preferably 0.5 mm or less, even more preferably 0.4 mm or less, even more preferably 0.3 mm or less.

In the present specification, the thickness of the outer peripheral wall 11 refers to a length in the normal direction of a side surface from the boundary between the outer peripheral wall 11 and the cell 13 or the partition wall 14 on the outermost side to the side surface of the honeycomb structure 10, in a cross-section orthogonal to the flow path direction of the honeycomb structure 10.

The length of the honeycomb structure 10 in the flow path direction and the cross-sectional area orthogonal to the flow path direction may be adjusted according to the required size of the heater element 1, 2, and are not particularly limited. For example, when used for heater element 1, 2 which are compact while ensuring a predetermined function, the honeycomb structure 10 may have a length of 2 to 20 mm in the flow path direction and a cross-sectional area orthogonal to the flow path direction of 10 cm² or more. The upper limit of the cross-sectional area orthogonal to the flow path direction is not particularly limited, but is, for example, 300 cm² or less.

The partition walls 14 constituting the honeycomb structure 10 is composed of a material capable of generating heat by energization, and specifically, is made of a material having PTC (Positive Temperature Coefficient) characteristics. If necessary, the outer peripheral wall 11 may also be made of a material having PTC characteristics similar to the partition walls 14.

It is possible to heat the functional material-containing layer 20 by heat transfer from the heated partition wall 14 (and the outer peripheral wall 11 if necessary). Further, the material having PTC characteristics has a characteristic that when the temperature rises and exceeds a Curie point, the resistance value rapidly rises and it becomes difficult for electricity to flow. Therefore, when the heater element 100 becomes hot, the current flowing through the partition wall 14 (and the outer peripheral wall 11 if necessary) is limited, so that excessive heat generation of the heater element 100 is suppressed. Therefore, it is also possible to suppress thermal deterioration of the functional material-containing layer 20 due to excessive heat generation.

The lower limit of the volume resistivity of the material having PTC characteristics at 25° C. is preferably 0.5 Ω·cm or more, more preferably 1 Ω·cm or more, and even more preferably 5 Ω·cm or more, from the viewpoint of obtaining appropriate heat generation. The upper limit of the volume resistivity of the material having PTC characteristics at 25° C. is preferably 20 Ω·cm or less, more preferably 18 Ω·cm or less, and even more preferably 16 Ω·cm or less, from the viewpoint of generating heat at a low drive voltage. In the present specification, the volume resistivity of a material having PTC characteristics at 25° C. is measured according to JIS K6271: 2008.

From the viewpoint of being able to generate heat when energized and having PTC characteristics, the outer peripheral wall 11 and the partition walls 14 are preferably made of a material containing barium titanate (BaTIO₃) as a main component, and more preferably ceramics made of a material containing barium titanate (BaTIO₃) based crystal particles in which a part of Ba is substituted with a rare earth element as a main component. In addition, in this specification, a “main component” means the component accounts for more than 50% by mass in the whole components. The content of BaTiO₃ crystal particles can be determined by fluorescent X-ray analysis. Other crystal particles can be measured in the same manner as this method.

The composition formula of the BaTiO₃-based crystal particles in which a part of Ba is replaced with a rare earth element can be expressed by (Ba₁₋ₓAₓ) TiO₃. In the composition formula, A represents one or more rare earth elements, and 0.0001≤x≤0.010.

A is not particularly limited as long as it is a rare earth element, but is preferably one or more selected from the group consisting of La, Ce, Pr, Nd, Eu, Gd, Dy, Ho, Er, Y and Yb, and it is more preferably La. x is preferably 0.001 or more, more preferably 0.0015 or more, from the viewpoint of suppressing the electric resistance from becoming too high at room temperature. On the other hand, x is preferably 0.009 or less from the viewpoint of suppressing insufficient sintering and excessively high electrical resistance at room temperature.

The content of BaTiO₃ crystal particles in which a part of Ba is replaced with a rare earth element is not particularly limited as long as it is the main component of the ceramics, but is preferably 90% by mass or more, more preferably 92% by mass or more, and even more preferably 94% by mass or more. In addition, the upper limit of the content of the BaTiO₃ crystal particles is not particularly limited, but is generally 99% by mass, preferably 98% by mass.

The content of the BaTiO₃ crystal particles can be measured by fluorescent X-ray analysis. Other crystal particles can be measured in the same manner as this method.

It is desirable that the materials used for the outer peripheral wall 11 and the partition walls 14 substantially contain no lead (Pb) from the viewpoint of reducing the environmental burden. Specifically, the outer peripheral wall 11 and the partition walls 14 preferably have a Pb content of 0.01% by mass or less, more preferably 0.001% by mass or less, and even more preferably 0% by mass. Due to the low Pb content, for example, the air heated by contacting the heated partition walls 14 can be safely applied to organisms such as humans. In the outer peripheral wall 11 and the partition walls 14, the Pb content is preferably less than 0.03% by mass, more preferably less than 0.01% by mass, and even more preferably 0% by mass, in terms of PbO. The lead content can be determined by ICP-MS (Inductively Coupled Plasma Mass Spectrometry).

The lower limit of the Curie point of the material constituting the outer peripheral wall 11 and the partition walls 14 is preferably 100° C. or higher, more preferably 110° C. or higher, and more preferably 125° C. or higher, from the viewpoint of efficiently heating air. In addition, regarding the upper limit of the Curie point, from the viewpoint of safety as a component placed in or near the vehicle compartment, it is preferably 250° C. or lower, more preferably 225° C. or lower, even more preferably 200° C. or lower, and even more preferably 150° C. or lower.

The Curie point of the material constituting the outer peripheral wall and the partition walls can be adjusted by the type of shifter and the amount of addition. For example, the Curie point of barium titanate (BaTIO₃) is about 120° C., but the Curie point can be shifted to the low temperature side by substituting a part of Ba and Ti with one or more of Sr, Sn and Zr.

In the present invention, the Curie point is measured by the following method. Attach the sample to a sample holder for measurement, mount it in a measuring tank (for example, MINI-SUBZERO MC-810P manufactured by ESPEC CORP.), and measure the change in the electrical resistance of the sample when the temperature is raised from 10° C. with a DC resistance meter (for example, Multimeter 3478A manufactured by YOKOGAWA HEWLETT PACKARD LTD). From the electric resistance-temperature plot obtained by the measurement, the temperature at which the resistance value becomes twice the resistance value at room temperature (20° C.) is defined as the Curie point.

### (1-2. Electrode)

In the heater element 1 according to the first embodiment of the present invention, the first electrode 30a is provided on the one end surface 12a. Further, the second electrode 30b has an electrode portion A provided on the other end surface 12b; and an electrode portion B connected to the electrode portion A and provided on the surface of the partition walls 14 over a predetermined length D1 from the other end surface 12b in the direction in which the flow paths extend.

In the heater element 1 according to the first embodiment, by arranging the first electrode 30a and the second electrode 30b in this way, the distance between the first electrode 30a and the second electrode 30b in the direction in which the flow paths extend can be shortened, compared to the case where the first electrode 30a and the second electrode 30b are provided only on the one end surface 12a and the other end surface 12b, respectively. Since the electrical resistance decreases as the distance between the electrodes becomes shorter, it becomes possible to widen the area in the direction in which the flow paths extend that can be effectively heated.

In the first embodiment, air may be caused to flow inside the cells 13 of the heater element 1 such that the one end surface 12a is on the upstream side and the other end surface 12b is on the downstream side, or it may be caused to flow inside the cell 13 of the heater element 1 such that the one end surface 12a is on the downstream side and the other end surface 12b is on the upstream side. However, while the upstream portion of the heater element 1 is cooled by the cold incoming air, the downstream portion is not cooled because the incoming air is heated. Therefore, since the downstream portion is sufficiently heated by heat conduction, even if no current flows through the honeycomb structure 10 in the downstream portion and electricity flows through the electrodes provided in the direction in which the flow paths extend, sufficient heating can be achieved. For this reason, it is preferable to cause the air to flow inside the cells 13 of the heater element 1 such that the one end surface 12a is on the upstream side and the other end surface 12b is on the downstream side, because the area in the direction in which the flow paths extend where the functional material-containing layer 20 can be effectively heated can be further expanded.

In the heater element 2 according to the second embodiment of the present invention, the first electrode 30a has an electrode portion A provided on the one end surface 12a, and an electrode portion B connected to the electrode portion A and provided on the surface of the partition walls 14 over a predetermined length D2a from the one end surface 12a in the direction in which the flow paths extend. Further, the second electrode 30b has an electrode portion A provided on the other end surface 12b, and an electrode portion B connected to the electrode portion A and provided on the surface of the partition wall 14 over a predetermined length D2b from the other end surface 12b in the direction in which the flow paths extend.

In the heater element 2 according to the second embodiment, by arranging the first electrode 30a and the second electrode 30b in this way, the distance between the first electrode 30a and the second electrode 30b in the direction in which the flow paths extend can be shortened, compared to the case where the first electrode 30a and the second electrode 30b are provided only on the one end surface 12a and the other end surface 12b, respectively. Since the electrical resistance decreases as the distance between the electrodes becomes shorter, it becomes possible to widen the area in the direction in which the flow paths extend that can be effectively heated.

In the second embodiment, air may be caused to flow inside the cells 13 of the heater element 1 such that the one end surface 12a is on the upstream side and the other end surface 12b is on the downstream side, or it may be caused to flow inside the cell 13 of the heater element 1 such that the one end surface 12a is on the downstream side and the other end surface 12b is on the upstream side. However, as mentioned above, in the downstream portion of the heater element 2, even if no current flows through the honeycomb structure 10 and electricity flows through the electrodes provided in the direction in which the flow paths extend, heating is still possible. For this reason, it is preferable to cause the air to flow inside the cells 13 of the heater element 1 such that the end surface provided with the electrode with the shorter average length of D2a and D2b is the upstream side, and the end surface provided with the electrode with the longer average length is the downstream side, because the area in the direction in which the flow paths extend where the functional material-containing layer 20 can be effectively heated can be further expanded.

In both the first embodiment and the second embodiment, the longer the predetermined lengths (D1, D2a, D2b) of the electrode portion B is, the shorter the distance between the electrodes is. Therefore, the predetermined lengths (D1, D2a, D2b) of the electrode portions B preferably have an average length of 1/200 or more, more preferably 1/100 or more, and even more preferably 1/50 or more of the length of the honeycomb structure 10 in the direction in which the flow paths extend. However, there is a limit to the distance that can be heated by heat conduction, and there is a risk that the electrode portion B of the first electrode 30a and the electrode portion B of the second electrode 30b may come into contact and cause a short circuit. For this reason, the predetermined lengths (D1, D2a, D2b) of the electrode portion B preferably have an average length of ½ or less, more preferably ⅓ or less, and even more preferably ¼ or less.

The average length of the electrode portion B in the direction in which the flow paths of the honeycomb structure 10 extend is measured by the following procedure. First, a cross-sectional image of the heater element is obtained at a magnification of approximately 50 times using a scanning electron microscope or the like. The cross section is a cross section parallel to the flow path direction and passing through the central axis O of the honeycomb structure 10 extending in the flow path direction, as illustrated in FIGS. 1C and 2C. The position of the central axis O is the center of gravity in the cross section orthogonal to the flow path of the honeycomb structure 10 (see FIGS. 1A and 2A). Next, when calculating the average length of D1 and D2b of the second electrode 30b, the lengths of all the electrode portions B of the second electrode 30b in the cross-sectional image from the other end surface 12b in the direction in which the flow paths of the honeycomb structure 10 extend are determined, and the average value is calculated. When calculating the average length of D2a of the first electrode 30a, the lengths of all the electrode portions B of the first electrode 30a in the cross-sectional image from the one end surface 12a in the direction in which the flow paths of the honeycomb structure 10 extend are determined, and the average value is calculated.

By applying a voltage between the first electrode 30a and the second electrode 30b, it becomes possible to cause the honeycomb structure 10 to generate heat using Joule heat. The first electrode 30a and the second electrode 30b may have an extension portion extending toward the outside of the honeycomb structure 10. Providing the extension portion facilitates connection with a connector responsible for connection with the outside.

The first electrode 30a and the second electrode 30b are not particularly limited, and for example, a metal or alloy containing at least one selected from Cu, Ag, Al, Ni and Si can be used. It is also possible to use an ohmic electrode capable of ohmic contact with the outer peripheral wall 11 and/or the partition walls 14 having PTC characteristics. As the ohmic electrode, for example, an ohmic electrode containing at least one selected from Al, Au, Ag and In as a base metal and at least one selected from Ni, Si, Ge, Sn, Se and Te for n-type semiconductors as a dopant can be used. Further, the first electrode 30a and the second electrode 30b may have a one-layer structure or a laminated structure with two or more layers. When the first electrode 30a and the second electrode 30b have a laminated structure of two or more layers, the materials of the respective layers may be the same type or different types.

The thicknesses of the first electrode 30a and the second electrode 30b are not particularly limited and can be appropriately set according to the method of forming the first electrode 30a and the second electrode 30b. Examples of the method for forming the first electrode 30a and the second electrode 30b include metal precipitation methods such as sputtering, vapor deposition, electrolytic precipitation, and chemical precipitation. Further, the first electrode 30a and the second electrode 30b may also be formed by a method of applying an electrode paste and then baking or may be formed by thermal spraying. Further, the first electrode 30a and the second electrode 30b may be formed by joining a metal plate or an alloy plate.

For either of the first electrode 30a or the second electrode 30b, the thickness of the electrode portion A is preferably about 5 to 30 μm in case of baking of electrode paste, about 100 to 1000 nm for dry plating such as sputtering and vapor deposition, about 10 to 100 μm for thermal spraying, and about 5 to 30 μm for wet plating such as electrolytic precipitation and chemical precipitation. Further, when joining a metal plate or an alloy plate, the thickness of the first electrode 30a and the second electrode 30b is preferably about 5 to 100 μm.

For either of the first electrode 30a or the second electrode 30b, it may be desirable that the average thickness of the electrode portion B be larger in terms of ensuring electrical continuity, but a smaller average thickness is advantageous in that the ventilation resistance of inflowing air can be reduced. Therefore, the average thickness of the electrode portion B is preferably 1/10000 or more and 1/10 or less, and more preferably 1/1000 or more and 1/20 or less of the hydraulic diameter of the cell 13. The hydraulic diameter of the cell 13 is a value (P−t) obtained by subtracting the partition wall thickness t (mm) from the cell pitch P (mm) described above.

The average thickness of each electrode portion B of the first electrode 30a and the second electrode 30b is measured by the following procedure. First, a cross-sectional image of the heater element is obtained at a magnification of approximately 50 times using a scanning electron microscope or the like. The cross section is a cross section parallel to the flow path direction and passing through the central axis O of the honeycomb structure 10 that extends in the flow path direction, as illustrated in FIGS. 1C and 2C. The position of the central axis O is the center of gravity in the cross section of the honeycomb structure 10 orthogonal to the flow path direction (see FIGS. 1D and 2D). For each electrode portion B visually recognized from the cross-sectional image, the average thickness is calculated by dividing the cross-sectional area by the length in the direction in which the flow path of the cell 13 extends. This calculation is performed for all electrode portions B of the first electrode 30a and second electrode 30b that are visually recognized from the cross-sectional image, and the overall average value is taken as the average thickness of each electrode portion B of the first electrode 30a and the second electrode 30b.

In either of the heater element 1 according to the first embodiment or the heater element 2 according to the second embodiment, the electrode portions B are continuously provided over the predetermined length on the entire surface of all the partition walls 14 partitioning the plurality of cells 13. In other words, in the area of the predetermined length, when the heater element 1, 2 is observed in a cross section orthogonal to the flow path direction, all of the partition walls 14 that partition the cells 13 (the partition walls 14 and outer peripheral wall 11 that partition the outermost cells 13) are covered over the entire circumference by the electrode portion B of the first electrode 30a or the second electrode 30b (See FIGS. 1D and 2D). With this configuration, the inter-electrode distance can be uniformly shortened in all cells 13. This makes it easier for the heater element 1, 2 to generate heat uniformly.

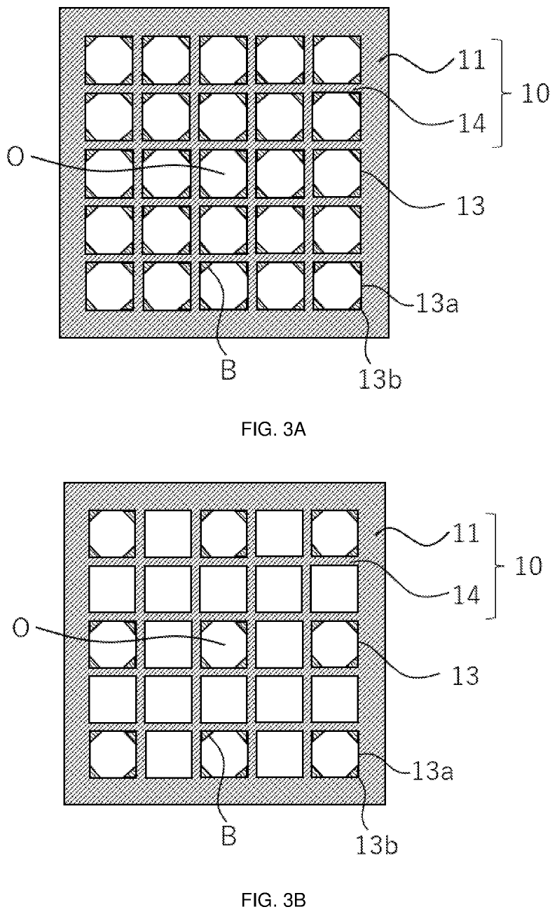

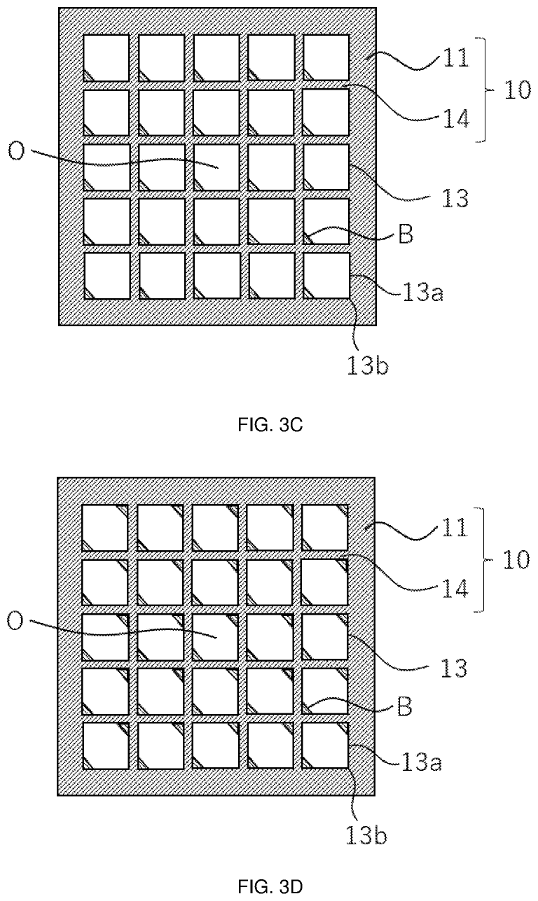

However, when observing the heater element 1, 2 in a cross section orthogonal to the flow path direction in the area of the predetermined length, the electrode portions B of the first electrode 30a and the second electrode 30b may include portions that do not cover the partition walls 14. That is, in another embodiment, the electrode portion B can be continuously provided over the predetermined length on a part of the surface of the partition walls 14 partitioning the plurality of cells 13. Such embodiments include: (1) an embodiment in which the electrode portion B is continuously provided over the predetermined length on a part of the surface of all the partition walls 14 partitioning the plurality of cells 13; and (2) an embodiment in which the electrode portion B is continuously provided over the predetermined length on a part of the surface or the entire surface of a part of the partition walls 14 partitioning the plurality of cells 13. FIGS. 3A to 3D show schematic diagrams of a cross section orthogonal to the flow path direction before forming a functional material-containing layer 20, for heater elements according to several other embodiments in which the structure of the electrode portion B of the first electrode 30a or the second electrode 30b is different.

In the embodiment of FIG. 3A, all cells 13 are provided with an electrode portion B. Further, the partition walls 14 that partition each cell 13 (in the case of the outermost cell 13, the partition walls 14 and outer peripheral wall 11 that partition the outermost cell 13) have a rectangular cross section, and all corner portions 13b are covered with the electrode portions B. On the other hand, none of the side portions 13a other than the corner portions 13b are covered with the electrode portion B.

In the embodiment of FIG. 3B, a part of cells 13 are provided with electrode portion B. Further, the partition walls 14 that partition each cell 13 in which the electrode portion B is provided (in the case of the outermost cell 13 where the electrode portion B is provided, the partition walls 14 and outer peripheral wall 11 that partition the outermost cell 13) have a quadrangular cross section, and all corner portions 13b are covered with the electrode portion B. On the other hand, none of the side portions 13a other than the corner portions 13b are covered with the electrode portion B. In addition, when providing the electrode portion B in a part of the cells 13, in the cross section orthogonal to the flow path direction, it is preferable from the viewpoint of heat generation uniformity to provide the electrode portions B point-symmetrically with respect to the central axis O, or to provide the electrode portions B line-symmetrically with respect to any line segment passing through the central axis O as the center of symmetry.

In the embodiment of FIG. 3C, all cells 13 are provided with an electrode portion B. Further, the partition walls 14 that partition each cell 13 (in the case of the outermost cell 13, the partition wall 14 and outer peripheral wall 11 that partition the outermost cell 13) have a quadrangular cross section, and only one corner portion 13b is covered with the electrode portion B. On the other hand, no portion other than one corner portion 13b is covered with the electrode portion B.

In the embodiment of FIG. 3D, all cells 13 are provided with electrode portions B. Further, the partition walls 14 that partition each cell 13 (in the case of the outermost cell 13, the partition walls 14 and outer peripheral wall 11 that partition the outermost cell 13) have a quadrangular cross section, and only a pair of opposing corner portions 13b are covered with the electrode portion B. On the other hand, no portion other than the pair of opposing corner portions 13b is covered with the electrode portion B.

### (1-3. Functional Material-Containing Layer)

A functional material-containing layer 20 can be provided on the surface of the partition walls 14 (in the case of the outermost cell 13, the partition walls 14 and outer peripheral wall 11 that partition the outermost cell 13) of the honeycomb structure 10. In addition to the partition wall 14, the functional material-containing layer 20 may be provided on the surface of at least one of the electrode portion B of the first electrode 30a and the electrode portion B of the second electrode 30b. It is more preferable that the functional material-containing layer 20 is provided at least on the surfaces of the partition walls 14 of the honeycomb structure 10 and the electrode portion B of the second electrode 30b. When the electrode portion B of the first electrode 30a exists, it is more preferable that the functional material-containing layer 20 is provided at least on the surfaces of the partition wall 14 of the honeycomb structure 10, the electrode portion B of the first electrode 30a, and the electrode portion B of the second electrode 30b.

The functional material contained in the functional material-containing layer 20 is not particularly limited as long as it is a material capable of exhibiting a desired function, but an adsorbent, a catalyst, or the like can be used. The adsorbent preferably has a function of adsorbing one or more kinds selected from components to be removed in the air, for example, water vapor, carbon dioxide, and an odor component. In addition, it is also preferable to have a function of adsorbing harmful volatile components. Further, by using a catalyst, the component to be removed can be purified. Further, an adsorbent and a catalyst may be used in combination for the purpose of enhancing the function of capturing the component to be removed by the adsorbent.

The adsorbent preferably has a function such that it is possible to adsorb components to be removed, such as water vapor, carbon dioxide, and harmful volatile components (for example, aldehydes, odor components, and the like) at −20 to 40° C., and release them at a high temperature of 60° C. or higher. Examples of the adsorbent having such a function include zeolite, silica gel, activated carbon, alumina, silica, low crystalline clay, and amorphous aluminum silicate complex, and the like. The type of the adsorbent may be appropriately selected according to the type of the component to be removed. One type of adsorbent may be used alone, or two or more types may be used in combination.

The catalyst preferably has a function capable of promoting a redox reaction. Examples of the catalyst having such a function include metal catalysts such as Pt, Pd and Ag, and oxide catalysts such as CeO₂ and ZrO₂. One type of catalyst may be used alone, or two or more types may be used in combination.

Harmful volatile components contained in the air of the vehicle compartment are, for example, volatile organic compounds (VOCs) and odor components. Specific examples of harmful volatile components include ammonia, acetic acid, isovaleric acid, nonenal, formaldehyde, toluene, xylene, paradichlorobenzene, ethylbenzene, styrene, chlorpyrifos, di-n-butyl phthalate, tetradecane, di-2-ethylhexyl phthalate, diazinon, acetaldehyde, N-methylcarbamic acid-2-(1-methylpropyl) phenyl, and the like.

The average thickness of the functional material-containing layer 20 may be determined according to the size of the cells 13, and is not particularly limited. For example, the average thickness of the functional material-containing layer 20 is preferably 20 μm or more, more preferably 25 μm or more, and even more preferably 30 μm or more, from the viewpoint of sufficiently ensuring contact with air. On the other hand, from the viewpoint of suppressing the peeling of the functional material-containing layer 20 from the partition walls 14 and the outer peripheral wall 11, the average thickness of the functional material-containing layer 20 is preferably 400 μm or less, more preferably 380 μm or less, and even more preferably 350 μm or less.

The thickness of the functional material-containing layer 20 is evaluated by the following procedure. As exemplified in FIG. 1C and FIG. 2C, an arbitrary cross-section that passes through a central axis O extending in the flow path direction of the honeycomb structure 10 and is parallel to the flow path direction is cut out, and a cross-sectional image of about 50 times is obtained with a scanning electron microscope or the like. The position of the central axis O is the position of the center of gravity in the cross-section orthogonal to the flow path direction of the honeycomb structure 10 (see FIG. 1D, FIG. 2D). For each functional material-containing layer 20 visually recognized from the cross-sectional image, the average thickness is calculated by dividing the cross-sectional area by the length in the flow path direction of the cells 13. This calculation is performed for all the functional material-containing layers 20 visually recognized from the cross-sectional image, and the overall average value is taken as the average thickness of the functional material-containing layer 20.

From the viewpoint that the functional material exerts a desired function in the heater element 100, the amount of the functional material-containing layer 20 is preferably 50 g/L or more and 500 g/L or less, more preferably 100 g/L or more and 400 g/L or less, and even more preferably 150 g/L or more and 350 g/L or less, with respect to the volume of the honeycomb structure 10. In addition, the volume of the honeycomb structure 10 is a value determined by the external dimensions of the honeycomb structure 10.

### (2. Method for Manufacturing Heater Element)

Next, a method for manufacturing a heater element according to the present invention will be exemplified.

A method for manufacturing a honeycomb structure constituting a heater element includes a forming step and a firing step.

In the forming step, a green body containing ceramic raw materials including BaCO₃ powder, TiO₂ powder, and rare earth nitrate or hydroxide powder is formed to prepare a honeycomb formed body having a relative density of 60% or more.

The ceramic raw material can be obtained by dry mixing each powder to obtain a desired composition.

The green body can be obtained by adding a dispersion medium, a binder, a plasticizer, and a dispersant to a ceramic raw material and kneading the mixture. The green body may contain additives such as a shifter, a metal oxide, a property improving agent, and a conductive powder, if necessary.

The blending amount of components other than the ceramic raw material is not particularly limited as long as the relative density of the honeycomb formed body is 60% or more.

Here, in this specification, the “relative density of the honeycomb formed body” means the ratio of the density of the honeycomb formed body to the true density of the entire ceramic raw material. Specifically, it can be determined by the following formula.

Relative density of honeycomb formed body (%)=Density of honeycomb formed body (g/cm³)/True density of entire ceramic raw material (g/cm³)×100

The density of the honeycomb formed body can be measured by the Archimedes method using pure water as a medium. Further, the true density of the entire ceramic raw material can be determined by dividing the total mass (g) of each raw material by the total solid volume (cm³) of each raw material.

Examples of the dispersion medium include water or a mixed solvent of water and an organic solvent such as alcohol, and water can be particularly preferably used.

Examples of the binder include organic binders such as methyl cellulose, hydroxy propyl methyl cellulose, hydroxy ethyl cellulose, carboxy methyl cellulose, and polyvinyl alcohol. In particular, it is suitable to use methyl cellulose and hydroxy propyl methyl cellulose in combination. One type of binder may be used alone, or two or more types may be used in combination, but it is preferable that the binder does not contain an alkali metal element.

Examples of the plasticizer include polyoxyalkylene alkyl ether, polycarboxylic acid polymer, and alkyl phosphate ester.

As the dispersant, surfactants such as polyoxyalkylene alkyl ether, ethylene glycol, dextrin, fatty acid soap, and polyalcohol can be used. As the dispersants, one type may be used alone, or two or more types may be used in combination.

A honeycomb formed body can be prepared by extrusion molding the green body. For extrusion molding, a die having a desired overall shape, cell shape, partition wall thickness, cell density, and the like can be used.

The relative density of the honeycomb formed body obtained by extrusion molding is 60% or more, preferably 65% or more. By controlling the relative density of the honeycomb formed body within such a range, it becomes possible to make the honeycomb formed body dense and reduce the electrical resistance at room temperature. In addition, the upper limit of the relative density of the honeycomb formed body is not particularly limited, but is generally 80%, and preferably 75%.

The honeycomb formed body may be dried before the firing step. The drying method is not particularly limited, and for example, conventionally known drying methods such as hot air drying, microwave drying, dielectric drying, reduced pressure drying, vacuum drying, and freeze drying can be used. Among them, a drying method that combines hot wind drying with microwave drying or dielectric drying is preferable because the entire formed body can be dried quickly and uniformly.

The firing step includes holding the temperature at 1150 to 1250° C., then raising the temperature to a maximum temperature of 1360 to 1430° C. at a heating rate of 20 to 600° C./hour, and holding the temperature for 0.5 to 10 hours.

By holding the honeycomb formed body at a maximum temperature of 1360 to 1430° C. for 0.5 to 10 hours, a honeycomb structure 10 mainly composed of BaTiO₃-based crystal particles in which a part of Ba is replaced with a rare earth element can be obtained.

Further, by maintaining the temperature at 1150 to 1250° C., Ba₂TiO₄ crystal particles generated during the firing step are easily removed, so that the honeycomb structure 10 can be made denser.

Furthermore, by setting the heating rate from 1150 to 1250° C. to the maximum temperature of 1360 to 1430° C. to 20 to 600° C./hour, 1.0 to 10.0% by mass of Ba₆Ti1₇O₄₀ crystal particles can be generated in the honeycomb structure 10.

The holding time at 1150 to 1250° C. is not particularly limited, but is preferably 0.5 to 10 hours. With such a holding time, Ba₂TiO₄ crystal particles generated during the firing step can be easily removed stably.

The firing step preferably includes holding the temperature at 900 to 950° C. for 0.5 to 5 hours. By holding the temperature at 900 to 950° C. for 0.5 to 5 hours, BaCO₃ is efficiently decomposed and a honeycomb structure 10 having a predetermined composition can be easily obtained.

In addition, a degreasing step for removing the binder may be performed before the firing step. The atmosphere in the degreasing step is preferably atmospheric air in order to completely decompose the organic components.

Furthermore, the atmosphere in the firing step is preferably atmospheric air from the viewpoint of controlling electrical characteristics and manufacturing cost.

The firing furnace used in the firing step and the degreasing step is not particularly limited, but an electric furnace, a gas furnace, or the like can be used.

By joining a pair of electrodes (first electrode 30a and second electrode 30b) to the honeycomb structure obtained in this way, a heater element can be manufactured. The electrode portion A of the first electrode 30a and second electrode 30b can be formed on the one end surface 12a and the other end surface 12b of the honeycomb structure 10, by metal deposition methods such as sputtering, vapor deposition, electrolytic deposition, and chemical deposition. Further, the electrode portion A can also be formed by applying an electrode paste on the one end surface 12a and the other end surface 12b of the honeycomb structure 10, and then by baking. Further, they can be formed by thermal spraying. The electrode portion A may be composed of a single layer, but it may be composed of a plurality of electrode layers having different compositions. When the electrode portion A is formed on the end surfaces by the above method, the cells can be prevented from being blocked by setting the thickness of the electrode layers so as not to be excessively large. For example, the thickness of the electrodes is preferably about 5 to 30 μm for baking a paste, about 100 to 1000 nm for dry plating such as sputtering and vapor deposition, about 10 to 100 μm for thermal spraying, and about 5 to 30 μm for wet plating such as electrolytic deposition and chemical deposition.

When the first electrode 30a and the second electrode 30b have both the electrode portion A and the electrode portion B, they can be formed, for example, by the following procedure. First, an electrode slurry containing an electrode material, an organic binder, and a dispersion medium is prepared, and the honeycomb structure is immersed in the slurry from the one end surface 12a or the other end surface 12b to a desired depth in the flow path direction of the honeycomb structure 10. The dispersion medium can be water, an organic solvent (example: toluene, xylene, ethanol, isopropanol, n-butanol, ethyl acetate, butyl acetate, terpineol, dihydroterpineol, texanol, ethylene glycol monobutyl ether acetate, diethylene glycol monoethyl ether acetate, diethylene glycol monoethyl ether, diethylene glycol monobutyl ether acetate, diethylene glycol monobutyl ether, propylene glycol monomethyl ether acetate, propylene glycol monomethyl ether), or a mixture thereof. Excess slurry on the outer periphery of the honeycomb structure 10 is removed by blowing and wiping. Thereafter, by drying the slurry, the electrode portion B can be formed on the surfaces of the partition walls 14 and the like, and the electrode portion A can be formed on the one end surface 12a or the other end surface 12b of the honeycomb structure 10. The electrode portion A may be formed separately by the method described above. Drying can be carried out, for example, by heating the heater element to a temperature of about 120 to 600° C. The series of steps of immersion, slurry removal, and drying may be performed only once, but by repeating the steps multiple times, the electrode portions A and B can be provided with desired thicknesses.

The surface tension changes depending on the viscosity of the slurry, and the state of coverage of the side portions 13a and corner portions 13b of the partition walls 14 partitioning the cells 13 (or the partition walls 14 and outer peripheral wall 11 that partition the outermost cell 13) by the electrode portions B can be changed. For example, when covering the entire surface of the partition wall 14 as shown in FIGS. 1D and 2D, the viscosity of the electrode slurry may be made relatively low. When coating only the corner portion 13b of the partition wall 14 as shown in FIGS. 3A to 3D, the viscosity of the electrode slurry may be made relatively high. The difference between the productions of FIGS. 3A to 3D can be realized by, for example, masking the one end surface 12a or the other end surface 12b of the honeycomb structure 10 when the honeycomb structure 10 is immersed in the electrode slurry. As a method of masking, for example, there is a method of attaching a resin sheet to the one end surface 12a or the other end surface 12b of the honeycomb structure 10, and using a laser to make holes in the resin sheet at locations corresponding to the cells 13 to which the electrode portions B are to be formed.

Next, a functional material-containing layer 20 is formed on the surfaces of the partition walls 14 and the like of the thus obtained heater element, thereby obtaining a heater element with a functional material-containing layer.

Although the method for forming the functional material-containing layer 20 is not particularly limited, it can be formed, for example, by the following steps. The heater element is immersed in a slurry containing a functional material, an organic binder, and a dispersion medium for a predetermined time, and excess slurry on the end surface and outer periphery of the honeycomb structure 10 is removed by blowing and wiping. The dispersion medium can be water, an organic solvent (example: toluene, xylene, ethanol, isopropanol, n-butanol, ethyl acetate, butyl acetate, terpineol, dihydroterpineol, texanol, ethylene glycol monobutyl ether acetate, diethylene glycol monoethyl ether acetate, diethylene glycol monoethyl ether, diethylene glycol monobutyl ether acetate, diethylene glycol monobutyl ether, propylene glycol monomethyl ether acetate, propylene glycol monomethyl ether), or a mixture thereof. Thereafter, the functional material-containing layer 20 can be formed on the surfaces of the partition walls 14 and the like by drying the slurry. Drying can be carried out, for example, by heating the heater element to a temperature of about 120 to 600° C. The series of steps of immersion, slurry removal, and drying may be performed only once, but by repeating them multiple times, the functional material-containing layer 20 of a desired thickness can be provided on the surface of the partition wall 14 and the like.

### (3. Vehicle Compartment Purification System)

According to one embodiment of the present invention, there is provided a vehicle compartment purification system comprising the above-mentioned heater element with a functional material-containing layer. The vehicle compartment purification system can be suitably used in various vehicles such as automobiles.

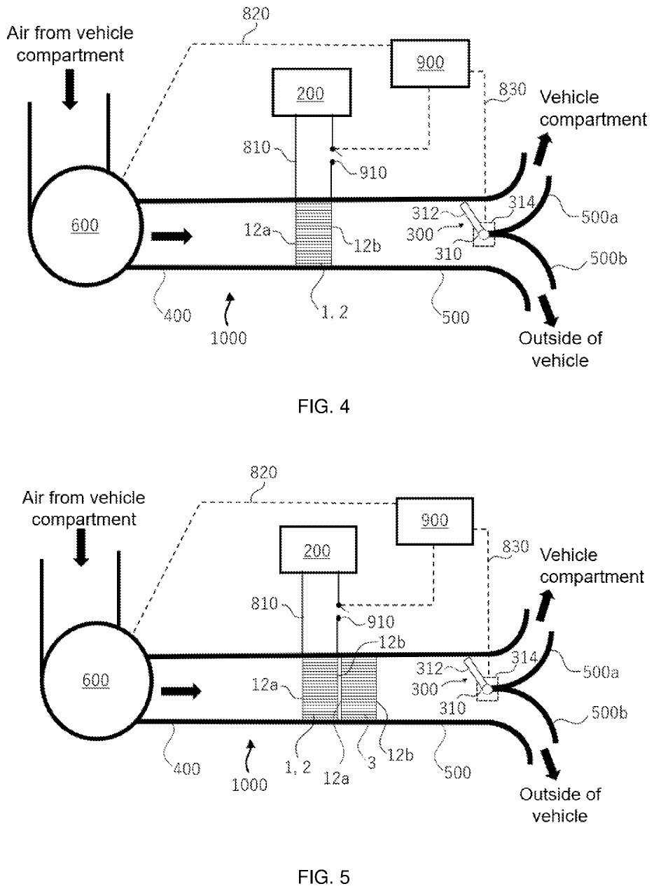

FIG. 4 is a schematic diagram showing the configuration of a vehicle compartment purification system according to an embodiment of the present invention.

The vehicle compartment purification system 1000 comprises:
at least one heater element 1, 2;
a power supply 200 for applying voltage to the heater element 1, 2;
an inflow piping 400 that communicates the vehicle compartment with an inlet end surface of the heater element 1, 2;
an outflow piping 500 having a first path 500a that communicates an outlet end surface of the heater element 1, 2 with the vehicle compartment; and
a ventilator 600 for causing air from the vehicle compartment to flow into the inlet end surface of the heater element via the inflow piping 400.

In the vehicle compartment purification system shown in FIG. 4, the heater element 1, 2 are arranged such that the one end surface 12a is the inlet end surface, and the other end surface 12b is the outlet end surface. However, the heater element 1, 2 can also be arranged such that the inlet end surface is the other end surface 12b and the outlet end surface is the one end surface 12a.

In addition to the first path 500a, the outflow piping 500 may have a second path 500b that communicates the outlet end surface of the heater element 1, 22 with the outside of the vehicle. Further, the outflow piping 500 can include a switching valve 300 that can switch the flow of air flowing through the outflow piping 500 between the first path 500a and the second path 500b.

The vehicle compartment purification system 1000 may have driving modes of:
a first mode in which the applied voltage from the power supply 200 is turned off, the switching valve 300 is switched such that the air flowing through the outflow piping 500 passes through the first path 500a, and the ventilator 600 is turned on, and
a second mode in which the applied voltage from the power supply 200 is turned on, the switching valve 300 is switched such that the air flowing through the outflow piping 500 passes through the second path 500b, and the ventilator 600 is turned on.

The vehicle compartment purification system 1000 may comprise a controller 900 capable of switching between the first mode and the second mode. For example, the controller 900 may be configured to be able to alternately execute the first mode and the second mode. By repeating switching between the first mode and the second mode in a constant cycle, it is possible to stably discharge the components to be removed from the vehicle compartment to the outside of the vehicle.

In the first mode, the vehicle compartment air is purified. Specifically, the air from the vehicle compartment passes through the inlet piping 400, flows into the inlet end surface of the heater element 1, 2, passes through the heater element 1, 2, and then flows out from the outlet end surface of the heater element 1, 2. Components to be removed from the air from the vehicle compartment are captured by the functional material while passing through the heater element 1, 2, and thereby removed. The clean air flowing out from the outlet end surface of the heater element 1, 2 is returned to the vehicle compartment through the first path 500a of the outflow piping 500.

In the second mode, the functional material is regenerated. Specifically, the air from the vehicle compartment passes through the inlet piping 400, flows into the inlet end surface of the heater element 1, 2, passes through the heater element 1, 2, and then flows out from the outlet end surface of the heater element 1, 2. The heater element 1, 2 generates heat when energized, which heats the functional material carried on the heater element 1, 2, so that the components to be removed, which are captured by the functional material, are desorbed from or reacts with the functional material.

In order to promote the desorption of the components to be removed captured by the functional material, it is preferable to heat the functional material to a temperature higher than the desorption temperature depending on the type of the functional material. For example, when an adsorbent is used as the functional material, it is preferable to heat at least a part, preferably the entire functional material to 70 to 150° C., more preferably 80 to 140° C., and even more preferably 90 to 130° C. Further, it is desirable that the second mode is performed for a time until the functional material is sufficiently regenerated. Although it depends on the type of the functional material, for example, when an adsorbent is used as the functional material, in the second mode, the functional material is preferably heated in the above temperature range for 1 to 10 minutes, more preferably heated for 2 to 8 minutes, and even more preferably heated 3 to 6 minutes.

The air from the vehicle compartment entraining the components to be removed that have been desorbed from the functional material while passing through the heater element 1, 2 flows out from the outlet end surface of the heater element 1, 2. The air containing the components to be removed that has flowed out from the outlet end surface of the heater element 1, 2 is discharged to the outside of the vehicle through the second path 500b of the outflow piping 500.

To switch the applied voltage to the heater element 1, 2 on and off, for example, the power supply 200 and the heater element 1, 2 can be electrically connected by an electric wire 810, and it is possible to operate a power switch 910 provided on the way. The controller 900 can execute the operation of the power switch 910.

To switch the ventilator 600 on and off, for example, the controller 900 and the ventilator 600 can be electrically connected by an electric wire 820 or wirelessly, and it is possible to operate a switch (not shown) of the ventilator 600 using the controller 900. The ventilator 600 can also be configured such that the amount of ventilation can be changed by the controller 900.

To switch the switching valve 300, for example, the controller 900 and the switching valve 300 can be electrically connected by an electric wire 830 or wirelessly, and it is possible to operate a switch (not shown) of the switching valve 300 using the controller 900.

The switching valve 300 is not particularly limited as long as it is a valve that is electrically driven and has a function of switching the flow paths, and examples thereof include an electromagnetic valve and an electric valve. In one embodiment, the switching valve 300 comprises an open/close door 312 supported by a rotary shaft 310, and an actuator 314 such as a motor that rotates the rotary shaft 310. The actuator 314 is configured to be controllable by the controller 900.

In the vehicle compartment purification system 1000, it is desirable that the heater element 100 is arranged at a position close to the vehicle compartment from the viewpoint of stably ensuring the above functions. Therefore, from the viewpoint of preventing electric shock, the drive voltage is preferably 60 V or less. Since the honeycomb structure 10 used in the heater element 100 has a low electrical resistance at room temperature, the honeycomb structure 10 can be heated with this low driving voltage. In addition, the lower limit of the drive voltage is not particularly limited, but is preferably 10 V or more. If the drive voltage is less than 10 V, the current at the time of heating the honeycomb structure 10 becomes large, so that it is necessary to make the electric wire 810 thick.

In the embodiment shown in FIG. 4, the ventilator 600 is installed on the upstream side of the heater element 1, 2. More specifically, the ventilator 600 is installed on the way of the inflow piping 400 that connects the heater element 1, 2 and the vehicle compartment, and the air that has passed through the ventilator 600 is pushed to flow into the heater element 1, 2. Alternatively, the ventilator 600 may be installed downstream of the heater element 1, 2. In this case, the ventilator 600 can be installed on the way of the outflow piping 500, for example, and the air that has passed through the inflow piping 400 is sucked to flow into the heater element 1, 2.

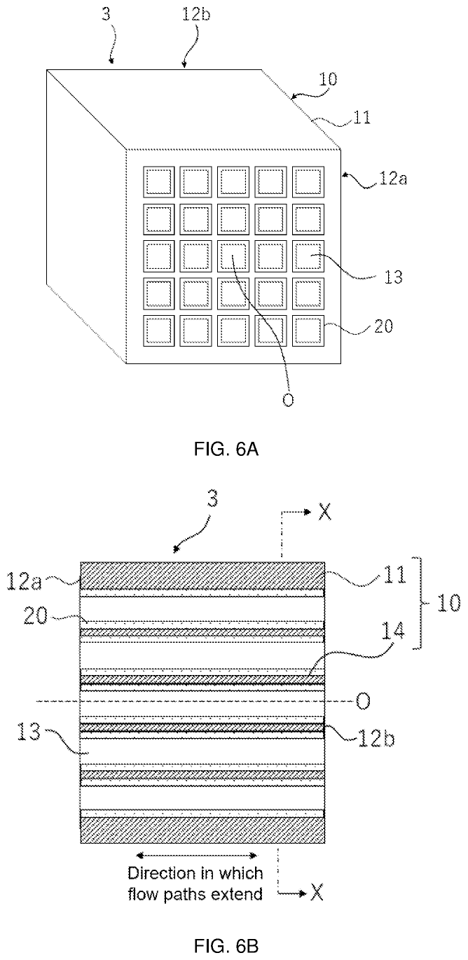

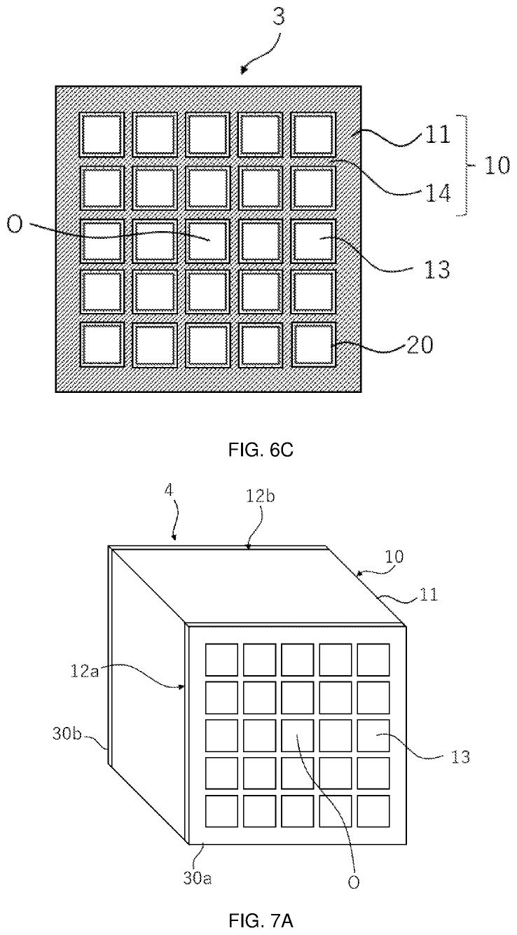

In another embodiment of the vehicle compartment purification system 1000, an additive functional body 3 may be arranged adjacent to the downstream side of the heater element 1, 2 (see FIG. 5). Referring to FIGS. 6A to 6C, in one embodiment, the additive functional body 3 comprises a honeycomb structure 10 comprising an outer peripheral wall 11 and partition walls 14 provided on the inner side of the outer peripheral wall 11, the partition walls 14 partitioning a plurality of cells 13 that form flow paths extending from one end surface 12a serving as the inlet end surface to the other end surface 12b serving as the outlet end surface.

The honeycomb structure 10 of the additive functional body 3 may have the same configuration as that described for the heater element 1, 2, including the shape and size of the honeycomb structure 10, the shape of the cells 13, the joining layer, the thickness of the partition walls 14, the cell density, the cell pitch (or the open frontal area of the cells), and the material. However, since the air heated by the heater element 1, 2 on the upstream side can flow into the additive functional body 3, the additive functional body 3 itself does not need to generate heat. Therefore, it is not necessary to provide a pair of electrodes in the additive functional body 3, and there is no need for the honeycomb structure 10 of the additive functional body 3 to be made of a material having PTC characteristics. Therefore, the honeycomb structure 10 of the additive functional body 3 can be manufactured using various ceramics. Among these, for reasons such as heat transfer and ease of manufacture, it is preferable that at least the partition walls 14 of the additive functional body 3 are made of cordierite.

In one embodiment, the additive functional body 3 may include a functional material-containing layer 20 provided on the surface of the partition walls 14 (in the case of the outermost cell 13, the partition walls 14 and outer peripheral wall 11 that partition the outermost cell 13). Although not limited, in the additive functional body 3, the functional material-containing layer 20 provided on the surface of the partition walls 14 of the honeycomb structure 10 may have the same configuration as that described for heater element 1, 2, including the type, average thickness, and amount of the functional material. When the additive functional body 3 is disposed adjacent to the heater element 1, 2 on the downstream side, the functional material-containing layer 20 does not need to be provided on the heater element 1, 2 on the upstream side. In addition, when a functional material-containing layer 20 is provided on the heater element 1, 2 which are on the upstream side, the additive functional body 3 on the downstream side may be provided with a functional material-containing layer 20 that can perform a function different from that of the functional material-containing layer 20 of the heater element 1, 2. Of course, the additive functional body 3 on the downstream side may also be provided with a functional material-containing layer 20 that can perform the same function as that of the functional material-containing layer 20 of the heater element 1, 2.

According to the vehicle compartment purification system 1000 according to the embodiment shown in FIG. 5, since the air can be heated by the heater element 1, 2 on the upstream side, there is no need to provide a pair of electrodes in the additive functional body 3 on the downstream side. Therefore, it is only necessary to consider the optimization of the functional material-containing layer 20 for the additive functional body 3, and the honeycomb structure 10 can be configured simply.

Expanding this idea further, even if the heater element installed on the upstream side cannot widen the area in the direction in which the flow paths extend where the functional material can be effectively heated, it can be understood that by arranging the additive functional body 3 adjacent to the downstream side of the heater element, it is possible to increase the proportion of the functional material that can be utilized effectively as a whole. In other words, the air already heated by the heater element on the upstream side can be allowed to flow into the additive functional body 3 on the downstream side, so there is no need to worry about the temperature near the inlet side of the additive functional body 3 becoming low. Therefore, the entire functional material included in the additive functional body 3 can be utilized effectively.

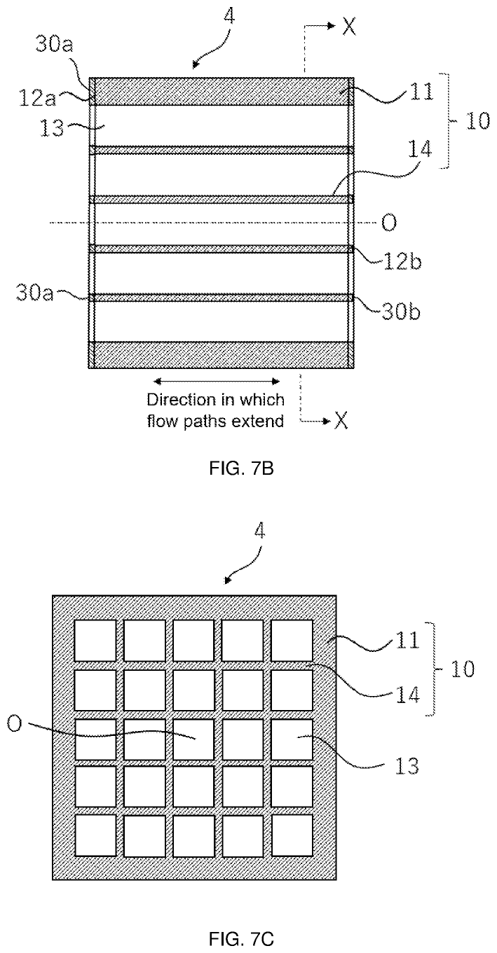

In this case, a functional material-containing layer may also be provided on the upstream heater element, but it is preferable not to provide it in order to increase the overall proportion of the functional material that can be utilized effectively. Furthermore, the upstream heater element that can be used in this case can adopt a simple electrode arrangement. FIGS. 7A to 7C show a schematic perspective view and a cross-sectional view of an example of a heater element 4 having such a simple electrode arrangement. The heater element 4 comprises a honeycomb structure 10 having an outer peripheral wall 11 and partition walls 14 provided on the inner side of the outer peripheral wall 11, the partition walls 14 partitioning a plurality of cells 13 that form flow paths extending from one end surface 12a serving as the inlet end surface to the other end surface 12b serving as the outlet end surface. Further, the heater element 4 comprises a first electrode 30a provided on one end surface 12a serving as the inlet end surface, and a second electrode 30b provided on the other end surface 12b serving as the outlet end surface.

The honeycomb structure 10 of the heater element 4 can have the same configuration as that described for the heater element 1, 2, including the shape and size of the honeycomb structure 10, the shape of the cells 13, the joining layer, the thickness of the partition walls 14, the cell density, the cell pitch (or the open frontal area of the cells), and the material, although it is not limited thereto. Further, the first electrode 30a and the second electrode 30b of the heater element 4 can have the same configuration as the electrode portion A described for the heater element 1, 2, including the material and thickness, although they are not limited thereto. In the heater element 4, there is no need to provide an electrode or a layer containing a functional material inside the cell 13. Therefore, the simple structure of the heater element 4 is also advantageous in reducing pressure loss when the air is caused to flow in the cell 13.

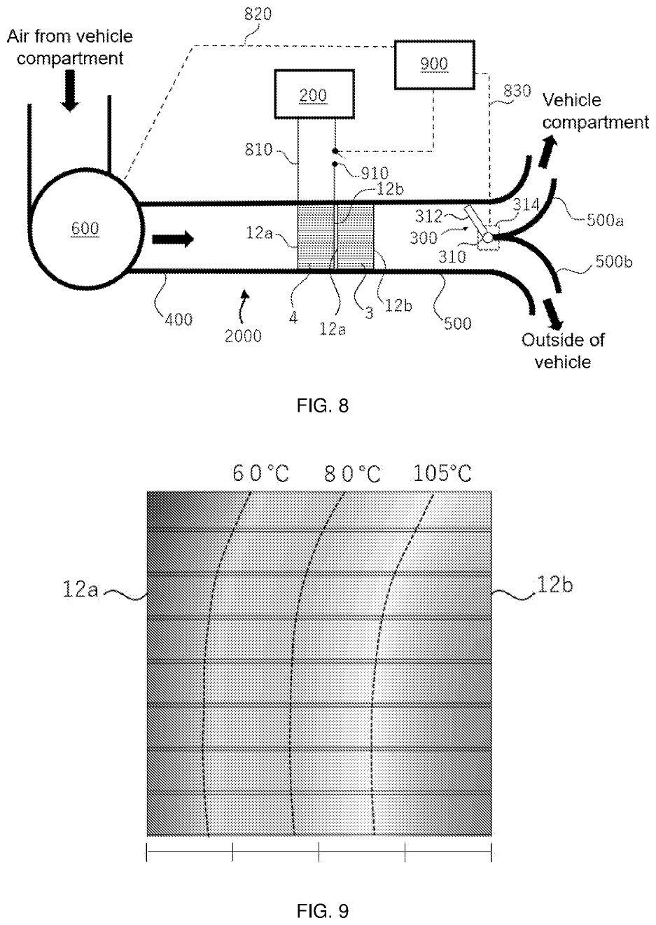

FIG. 8 is a schematic diagram showing the configuration of a vehicle compartment purification system 2000 according to yet another embodiment of the present invention based on the above concept.

The vehicle compartment purification system 2000 comprises:
a heater element 4;
an additive functional body 3 disposed adjacent to the downstream side of the heater element 4;
a power supply 200 for applying voltage to the heater element 4;
an inflow piping 400 that communicates the vehicle compartment with one end surface 12a serving as the inlet end surface of the heater element 4;
an outflow piping 500 having a first path 500a that communicates the other end surface 12b serving as the outlet end surface of the additive functional body 3 with the vehicle compartment; and
a ventilator 600 for causing air from the vehicle compartment to flow into one end surface 12a serving as the inlet end surface of the heater element 4 via the inflow piping 400.

The other configurations and operation modes of the vehicle compartment purification system 2000 are the same as those described for the vehicle compartment purification system 1000, and therefore the description thereof is omitted.

### (4. Simulation)

The results of simulating the temperature distribution inside the honeycomb structure when heat is generated while flowing air from one end surface to the other end surface of the honeycomb structure are shown.

### [Specifications of Honeycomb Structure]

The specifications of the honeycomb structure used in the simulation were as follows.
Shape of the cross section and end surface of the honeycomb structure orthogonal to the flow path direction: square
Cell shape orthogonal to flow path direction: square
Thickness of partition walls: 0.1016 mm
Cell density: 62 cells/cm²
Cell pitch: 1.270 mm
Cell open frontal area: 0.85
Size of cross section orthogonal to the flow path direction of the honeycomb structure: 10 mm×0.635 mm
Length of honeycomb structure in flow path direction: 10 mm
Volume resistivity at 25° C. of the material constituting the outer peripheral wall and partition walls: 14 Ω·cm (almost no change up to 120° C.)
Curie point of the material constituting the outer peripheral wall and partition walls: 120° C. (assuming barium titanate)
Density of materials composing the outer peripheral wall and partition walls: 4500 kg/m3
Specific heat of material composing the outer peripheral wall and partition walls: 590 J/kg/K

### [Heating Test]

A heating test was simulated to investigate the steady state temperature distribution inside the honeycomb structure when air (initial temperature=20° C.) is caused to flow through the cells of the honeycomb structure from one end surface to the other end surface at a rate of 0.13 m/sec, while applying a constant voltage of 12V between the one end surface and the other end surface of the honeycomb structure. Fluent Ver2021-R1 (provided by Ansys, Inc.) was used for the simulation.

The results are shown in FIG. 9. From this result, it can be understood that in the area approximately ¼ of the length of the honeycomb structure from the inlet side (the one end surface), even if a functional material is carried, since it is difficult to heat it to 60° C. or higher, which is advantageous for regeneration, and it cannot be utilized effectively. On the other hand, it can be seen that in the area approximately ¼ of the length of the honeycomb structure from the outlet side (the other end surface) it is heated to a temperature of 100° C. or higher, and in the area of about ½, it is heated to a temperature of 80° C. or higher.

Therefore, it can be understood that, by shortening the distance between the electrodes, it is advantageous to reduce the electrical resistance between the electrodes and to widen the area in the direction in which the flow paths extend that can be utilized effectively. Further, when shortening the distance between electrodes, it can be understood that, to effectively heat the inlet side, which is difficult to heat, it is better to provide the electrode portion B only on the outlet side, which can be heated more easily, or when providing electrode portions B on both the inlet and outlet sides, it is better to make the electrode portion B on the outlet side longer.

### DESCRIPTION OF REFERENCE NUMERALS

1: Heater element
2: Heater element
3: Additive functional body
4: Heater element
10: Honeycomb structure
11: Outer peripheral wall
12a: One end surface
12b: The other end surface
13: Cell
13a: Side portion
13b: Corner portion
14: Partition wall
20: Functional material-containing layer
30a: First electrode
30b: Second electrode
200: Power supply
300: Switching valve
310: Rotary shaft
312: Open/close door
314: Actuator
400: Inflow piping
500: Outflow piping
500a: First path
500b: Second path
600: Ventilator
810: Electric wire
820: Electric wire
830: Electric wire
900: Controller
910: Power switch
1000: Vehicle compartment purification system
2000: Vehicle compartment purification system

1. A heater element, comprising:
a honeycomb structure comprising an outer peripheral wall and partition walls provided on an inner side of the outer peripheral wall, the partition walls partitioning a plurality of cells that form flow paths extending from one end surface to an other end surface, wherein at least the partition walls are made of a material having PTC characteristics; and
a pair of electrodes composed of a first electrode and a second electrode;
wherein the first electrode and the second electrode satisfy either of the following condition (i) or (ii):
(i) the first electrode is provided on the one end surface, and
the second electrode comprises an electrode portion A provided on the other end surface, and an electrode portion B connected to the electrode portion A and provided on a surface of the partition walls over a predetermined length from the other end surface in a direction in which the flow paths extend;
(ii) the first electrode comprises an electrode portion A provided on the one end surface, and an electrode portion B connected to the electrode portion A and provided on the surface of the partition walls over a predetermined length from the one end surface in the direction in which the flow paths extend, and
the second electrode comprises an electrode portion A provided on the other end surface, and an electrode portion B connected to the electrode portion A and provided on a surface of the partition walls over a predetermined length from the other end surface in a direction in which the flow paths extend.

2. The heater element according to claim 1, wherein the predetermined length of the electrode portion B has an average length of 1/200 or more and less than ½ of a length of the honeycomb structure in the direction in which the flow paths extend.

3. The heater element according to claim 1, wherein the electrode portion B is continuously provided over the predetermined length over the entire surface of all the partition walls partitioning the plurality of cells.

4. The heater element according to claim 1, wherein the electrode portion B is continuously provided over the predetermined length on a part of the surface of the partition walls partitioning the plurality of cells.

5. The heater element according to claim 1, wherein the material having PTC characteristics is composed of a material containing barium titanate as a main component and substantially free of lead.

6. The heater element according to claim 1, wherein a volume resistivity at 25° C. of the material having PTC characteristics is 0.5 Ω·cm or more and 20 Ω·cm or less.

7. The heater element according to claim 1, wherein an average thickness of the electrode portion B is 1/10,000 or more and 1/10 or less of a hydraulic diameter of the cells.

8. The heater element according to claim 1, wherein in the honeycomb structure, a thickness of the partition walls is 0.125 mm or less, a cell density is 100 cells/cm² or less, and a cell pitch is 1.0 mm or more.

9. The heater element according to claim 1, wherein in the honeycomb structure, a thickness of the partition walls is 0.08 mm or more and 0.36 mm or less, a cell density is 2.54 cells/cm² or more and 140 cells/cm² or less, and an open frontal area of the cells is 0.70 or more.

10. The heater element according to claim 1, wherein the first electrode and the second electrode are made of the same material.

11. The heater element according to claim 1, further comprising a functional material-containing layer on the surface of the partition walls.

12. The heater element according to claim 11, wherein the functional material-containing layer contains a functional material having a function of adsorbing one or more selected from water vapor, carbon dioxide, and odor components.

13. The heater element according to claim 11, wherein the functional material-containing layer comprises a catalyst.

14. A vehicle compartment purification system, comprising:
at least one heater element according to claim 1;
a power supply for applying voltage to the heater element;
an inflow piping that communicates the vehicle compartment with an inlet end surface of the heater element;
an outflow piping having a first path that communicates an outlet end surface of the heater element with the vehicle compartment;
a ventilator for causing air from the vehicle compartment to flow into the inlet end surface of the heater element via the inflow piping;
wherein the heater element is arranged such that the inlet end surface is the one end surface and the outlet end surface is the other end surface, or such that the inlet end surface is the other end surface, and the outlet end surface is the one end surface.

15. The vehicle compartment purification system according to claim 14, wherein the heater element is arranged such that the inlet end surface is the one end surface and the outlet end surface is the other end surface.

16. The vehicle compartment purification system according to claim 14,
wherein in addition to the first path, the outflow piping comprises a second path that communicates the outlet end surface of the heater element with an outside of the vehicle, and
the outflow piping comprises a switching valve that is configured to switch a flow of the air flowing through the outflow piping between the first path and the second path;
wherein the vehicle compartment purification system further comprises a controller capable of switching between:
a first mode in which the applied voltage from the power supply is turned off, the switching valve is switched such that the air flowing through the outflow piping passes through the first path, and the ventilator is turned on, and
a second mode in which the applied voltage from the power supply is turned on, the switching valve is switched such that the air flowing through the outflow piping passes through the second path, and the ventilator is turned on.

17. The vehicle compartment purification system according to claim 14, wherein an additive functional body is provided downstream and adjacent to the heater element, the additive functional body comprising:
a honeycomb structure comprising an outer peripheral wall and partition walls provided on an inner side of the outer peripheral wall, the partition walls partitioning a plurality of cells that form flow paths extending from an inlet end surface to an outlet end surface; and
a functional material-containing layer provided on a surface of the partition walls.

18. The vehicle compartment purification system according to claim 17, wherein at least the partition walls of the additive functional body is made of cordierite.

19. A vehicle compartment purification system, comprising:
a heater element comprising:
a honeycomb structure comprising an outer peripheral wall and partition walls provided on an inner side of the outer peripheral wall, the partition walls partitioning a plurality of cells that form flow paths extending from an inlet end surface to an outlet end surface;
a first electrode provided on the inlet end surface; and
a second electrode provided on the outlet end surface;
an additive functional body provided downstream and adjacent to the heater element, the additive functional body comprising:
a honeycomb structure comprising an outer peripheral wall and partition walls provided on an inner side of the outer peripheral wall, the partition walls partitioning a plurality of cells that form flow paths extending from an inlet end surface to an outlet end surface; and
a functional material-containing layer provided on a surface of the partition walls;
a power supply for applying voltage to the heater element;
an inflow piping that communicates the vehicle compartment with the inlet end surface of the heater element;
an outflow piping comprising a first path that communicates the outlet end surface of the additive functional body with the vehicle compartment; and
a ventilator for causing air from the vehicle compartment to flow into the inlet end surface of the heater element via the inflow piping.
